(19) United States
(12) Patent Application Publication
(10) Pub. No.: US 2026/0229368 A1
(43) Pub. Date: Aug. 6, 2026

(54) SYSTEM AND METHOD FOR GENERATING AND ASSESSING DATA AGGREGATIONS FOR HEALTHCARE

(71) Applicant: BranchLab Inc., Boulder, CO (US)

(72) Inventors: Joshua Walsh, Boulder, CO (US); Christopher Cagle, Pittsburgh, PA (US)

(73) Assignee: BranchLab Inc., Boulder, CO (US)

(21) Appl. No.: 19/529,842

(22) Filed: Feb. 4, 2026

Related U.S. Application Data
(60) Provisional application No. 63/753,654, filed on Feb. 4, 2025.

(51) Int. Cl. G16H 50/70; G06F 21/62; G16H 50/50

(52) U.S. Cl. CPC G16H 50/70; G06F 21/6254; G16H 50/50

(57) ABSTRACT

Disclosed are systems and methods for generating and assessing data aggregations including defining audience characteristic data; querying a health database including health information and a health context database including health context information; aggregating the information into a set of data segments organized by age, gender, and geographic location, where the set of de-identified audience data segments is devoid of personally identifiable information (PII), including at least a count for the segment and values for the individual data points; assigning each of the data segments to an age bucket; calculating a probability distribution for the set of age buckets; calculating a probability score for each of the age buckets in the set using the probability distribution; querying a target database and appending the calculated probability score of the age bucket to one or more target data entries in the target database.

### CROSS-REFERENCE TO RELATED APPLICATIONS

This U.S. Non-Provisional Application is related to and claims priority to U.S. Provisional Application No. 63/753,654, filed on Feb. 4, 2025, the entire contents of which are incorporated herein by reference.

### FIELD

The subject matter disclosed relates generally to computer implementations of generating and assessing data aggregations in a computer system, and in some embodiments, to methods, systems, and non-transitory computer readable media encoded with program code for generating and assessing data aggregations including healthcare data.

### BACKGROUND INFORMATION

In many instances, large volumes of distributed, unrelated datasets, stored in various different data formats, may cause difficulty querying multiple databases and data stored in the various databases. Additionally, some data formats, for example those mandated by different organizations or industries, such as healthcare rules and regulations, must be adhered to by some of the datasets, but likely not all available datasets, therefor causing inconsistencies in various datasets and making merging and aggregating data from different sources challenging and time consuming. In instances where data could be aggregated and merged, the data may be difficult to query because of such differences and inconsistencies between the datasets. It may also be difficult to use the data for training machine learning models, as the large amounts of data may result in inconsistently trained models if used for model training. Even further, no methods exist to gather data from various datasets, databases, and/or data sources with data that is completely free of personally identifiable information (PII) once merged and/or synthesized. Such data should be anonymized and generalized, but is still required to be understandable for training machine learning algorithms and other data analysis purposes. Higher level representations of the data do not exist for more generalized training of machine learning models or targeted training for specific use cases.

### SUMMARY

Embodiments may relate to a computer-implemented method for generating and assessing data aggregations. The method may include defining audience characteristic data. The audience characteristic data may include at least an age range and a medical condition. The method may include querying a health data database including health data entries. Each health data entry may include at least an age field, a gender field, and a geographic location filed. The audience characteristic data can be received as health information including a set of individual data points paired with an age value, a gender character, and a geographic location value which can be correlated with the health information. The method may include querying a health context database including health context data entries. Each health context data entry may include at least an age field, a gender field, and a geographic location field. The audience characteristic data can be received as health context information including a set of individual data points paired with an age value, a gender character, and a geographic location value which can be correlated with the health context information. The method may include querying an online activity database including online activity data entries. Each online activity data entry may include at least an age, a gender, and a geographic location field. The audience characteristic data can be received as online activity information including a set of individual data points paired with an age value, a gender character, and a geographic location value which can be correlated with the online activity information. The method may include aggregating the health information received from the health data database, the health context information received from the health context database, and the online activity information received from the online activity database into a set of de-identified audience data segments organized by age, gender, and geographic location. The set of de-identified audience data segments can be devoid of personally identifiable information (PII). Each de-identified audience data segment in the set may include at least a count for the segment and values for the individual data points. The method may include assigning each of the de-identified audience data segments of the set to an age bucket within a set of age buckets. The age buckets can be defined by an age range and a gender. The method may include calculating a probability distribution for the set of age buckets. The method may include calculating a probability score for each of the age buckets in the set using the probability distribution and weights assigned to the individual data points. The method may include querying a target database, keyed by at least age, gender, and geographic location, and appending the calculated probability score of the age bucket to one or more target data entries in the target database that matches with the age, gender, and geographic information from the target database. The method may include transmitting updated health information to one or more client devices associated with at least one audience data segment of the set of de-identified audience data segments such that the updated health information is targeted based on relevance to users of the client devices without being associated to any PII.

Embodiments may relate to a system for managing data aggregations and annotating target databases. The system may include at least one health data database, at least one health context database, and at least one online activity database. The system may include a memory storing program code for an audience definition module, a database query module, and a modeling module. The system may include a processor configured to execute program code. The program code for the audience definition module, when executed, can cause the processor to receive text input defining an audience definition. The text input may include at least an age range and a medical condition. The program code for the audience definition module, when executed, can cause the processor to structure the text input into an audience data object in an audience file including at least an age range field and a medical condition field. The program code for the database query module, when executed, can cause the processor to query a health data database to extract corresponding health data entries based on the audience file. The program code for the database query module, when executed, can cause the processor to query a health context database to extract corresponding health context data entries based on the audience file. The program code for the database query module, when executed, can cause the processor to query an online activity database to extract corresponding online activity data entries based on the audience file. The program code for the database query module, when executed, can cause the processor to synthesize the extracted corresponding health data entries, corresponding health context data entries, and corresponding online activity data entries to generate de-identified aggregate data in an aggregate data file. The program code for the modeling module, when executed, can cause the processor to clean the de-identified aggregate data to generate consistent data fields within the aggregate data file. The program code for the modeling module, when executed, can cause the processor to automatically cluster the de-identified aggregate data into plural segments based on an age value. The program code for the modeling module, when executed, can cause the processor to calculate plural joint probability values based on the plural segments, and at least the age ranges and the medical conditions defined by aggregated audience data objects. The program code for the modeling module, when executed, can cause the processor to calculate a final probability score for each of the plural segments using the plural joint probability values and weights assigned to each field in the audience data objects. The program code for the modeling module, when executed, can cause the processor to query a target database including data entries with at least an age field, a gender field, and a geographic location field. The program code for the modeling module, when executed, can cause the processor to append each of the final probability scores calculated for each of the plural segments to a target data entry in the target database, wherein the segment matches with the age field, the gender field, and the geographic information field from the target data entry in the target database. The program code for the modeling module, when executed, can cause the processor to transmit updated health information to one or more client devices associated with at least one segment such that the updated health information is transmitted to users where the updated health information can be useful to the client devices without being associated to any PII of the users.

### BRIEF DESCRIPTION OF THE DRAWINGS

Other objects and advantages of the present disclosure will become apparent to those skilled in the art upon reading the following detailed description of exemplary embodiments, in conjunction with the accompanying drawings, in which like reference numerals have been used to designate like elements, and in which:

FIG. 1 is a diagram of an exemplary system configuration for generating and assessing data aggregations and/or annotating target databases as disclosed herein;

FIG. 2 is a flow diagram of an exemplary method for managing data aggregations and/or annotating target databases as disclosed herein;

FIG. 3 is a flow diagram of an exemplary system and/or components pipeline for generating and assessing data aggregations and/or annotating target databases disclosed herein;

FIG. 4 is a diagram of an exemplary environment in which methods, systems, and/or computer program products, described herein, may be implemented as disclosed herein;

FIG. 5 is a diagram of an exemplary architecture for a computer system and/or network in which exemplary system configurations for generating and assessing data aggregations and/or annotating target databases may be implemented as disclosed herein;

FIG. 6 is a diagram of an exemplary audience file including exemplary audience data objects for generating and assessing data aggregations and/or annotating target databases disclosed herein;

FIG. 7 is a diagram of exemplary de-identified aggregate data in an exemplary aggregate data file for generating and assessing data aggregations and/or annotating target databases as disclosed herein;

FIG. 8 is a flow diagram for an exemplary method performed by a modeling module configured with program code for generating and assessing data aggregations and/or annotating target databases as disclosed herein;

FIGS. 9A-9D are diagrams of exemplary database entries having appended final probability scores as disclosed herein; and

FIG. 10 is a schematic diagram of example components of one or more systems and/or devices of FIG. 1 and/or FIG. 4 as disclosed herein.

### DETAILED DESCRIPTION

Embodiments disclosed herein present a novel approach to aggregating and assessing various health related data points from users of client devices. The various health related data points can be aggregated and/or assessed from third-party and/or client databases. For example, embodiments disclosed herein can improve utility of third-party database and data stored in third-party databases by aggregating data from other data source to generate scores that can be appended to the data in the third-party databases. The scores appended to the data in the third-party databases can allow third-parties (or other parties and/or devices) to determine which data entries are more relevant for a specific purpose based on the generated and appended score. Embodiments disclosed herein may provide for streamlined analysis of data from various data sources, while also combining health data context with online activity and other health factors of users from various storage locations. Such embodiments may result in reduced storage of data and increased relevancy of updated health information transmitted to users of client devices. Embodiments including specially configured processors can allow for detection of commonalities across health data, without the need for PII within the data. Further, some embodiments may allow for the removal and/or non-use of PII in data where PII is present, while providing insights into users of client devices that can be used to provide updated health information based on scored and/or annotated target databases without having to use and/or analyze PII of the users. Such embodiments increase coverage of updated health information while improving security of PII and improving privacy of the various users connected to a network.

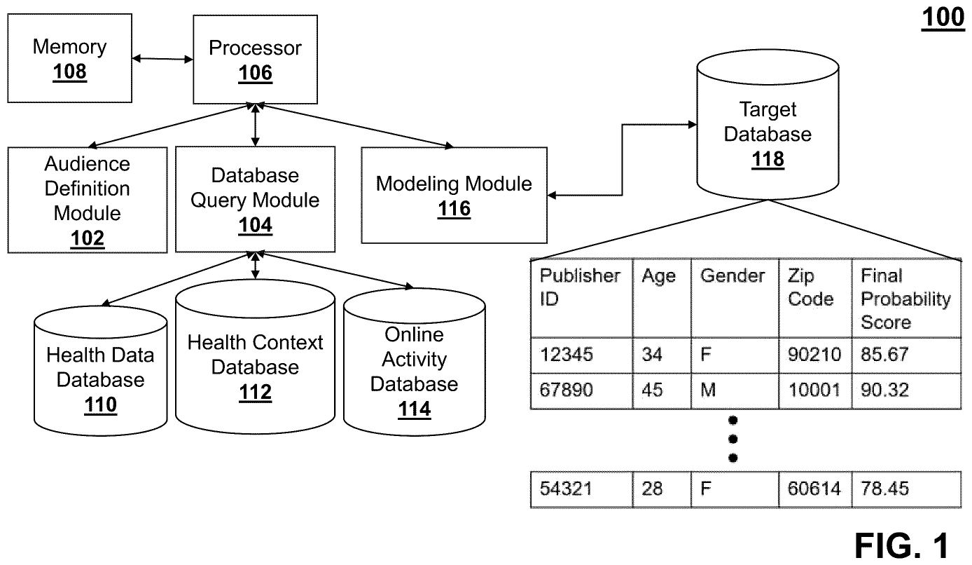

FIG. 1 shows a diagram of an exemplary system configuration for generating and assessing data aggregations and/or annotating target databases as disclosed herein. The various components of FIG. 1 can be implemented in and/or processed by a specially configured processor (e.g., a CPU) and/or on any number of specially configured distributed processors (e.g., a distributed and/or decentralized computing system) coupled with memory and connected via a communications network. Each of the components shown in FIG. 1 are described in the context of an exemplary embodiment.

As shown in FIG. 1, embodiments relate to a computing system 100 configured for generating and assessing data aggregations and/or annotating target databases. In some embodiments, computing system 100 can be specially configured for generating and assessing data aggregations and/or annotating target databases within a computing network. Computing system 100 can include audience definition module 102, database query module 104, processor 106, memory 108, health data database 110, health context database 112, online activity database 114, modeling module 116, and target database 118.

Computing system 100 can be configured for generating and assessing data aggregations and annotating target databases. Computing system 100 may include at least one health data database, at least one health context database, and at least one online activity database. For example, computing system 100 may include health data database 110, health context database 112, and online activity database 114.

Computing system 100 may include a memory storing program code for an audience definition module, a database query module, and a modeling module. For example, computing system 100 may include memory 108 storing program code for audience definition module 102, database query module 104, and modeling module 116.

Computing system 100 may include a processor configured to execute program code. For example, computing system 100 may include processor 106 configured to execute program code for audience definition module 102, database query module 104, and modeling module 116. The program code for the audience definition module, when executed by the processor, can cause the processor to receive text input defining an audience definition, wherein the text input includes at least an age range and a medical condition. For example, processor 106 may execute program code for audience definition module 102, wherein audience definition module 102 may cause processor 106 to receive text input data defining an audience definition. The text input may include at least an age range and a medical condition. In some embodiments, the text input can include any one or more of the following: an age, a gender, a state, a market area, a timeframe, a diagnosis, a drug, a treatment, an insurance provider, a medical condition, and/or a payer type. Text input may be provided by a user and may be analyzed by a large language model (LLM) to define various attributes of the audience definition, such as the age range and the medical condition, based on the text input data. In this way, the LLM may analyze the text input data to identify at least one of an age, a gender, a state, a market area, a timeframe, a diagnosis, a drug, a treatment, an insurance provider, a medical condition, and/or a payer type defined within the text input data.

In some embodiments, the text input can include an insurance provider and a geographic location. The audience data object can include an insurance provider field and a geographic location field. The insurance provider in the insurance provider field in the audience data object is replaced with an insurance provider code and the geographic location in the geographic location field in the audience data object is replaced with a geographic location code.

The program code for the audience definition module, when executed by the processor, can cause the processor to structure the text input into an audience data object in an audience file including at least an age range field and a medical condition field. For example, processor 106 may execute program code for audience definition module 102, wherein audience definition module 102 may cause processor 106 to structure the text input data into an audience data object in an audience file. The audience data object may include at least an age range field and a medical condition field to store the age range and medical definition corresponding to the text input data. In some embodiments, the audience data object can be stored in JavaScript Object Notation (JSON) in the audience file. In some embodiments, the medical condition and/or the data within the medical condition field can be defined using an International Classification of Diseases (ICD) code (e.g., an ICD-10 code). In some embodiments, the medical condition in the medical condition field in the audience data object can be replaced with an ICD-10 code or a National Drug Code (NDC) associated with the medical condition. In some embodiments, the medical condition in the medical condition field in the audience data object can be replaced with any one of an International ICD-10 code, a NDC associated with the medical condition, and/or a current procedural terminology (CPT) code.

The program code for the database query module, when executed by the processor, can cause the processor to query a health data database to extract corresponding health data entries based on the audience file. For example, processor 106 may execute program code for database query module 104, wherein database query module 104 may cause processor 106 to query health data database 110 to extract corresponding health data entries based on the audience file. Processor 106 may determine that the corresponding health data entries include an age field with an age value that is within the age range of the audience definition and that the corresponding health data entries include a medical condition field with a medical condition value that matches or closely matches the medical condition of the audience definition. In this way, processor 106 and database query module 104 may precisely identify and pinpoint data entries that may be directly relevant to and/or match the audience definition.

The program code for the database query module, when executed by the processor, can cause the processor to query a health context database to extract corresponding health context data entries based on the audience file. For example, processor 106 may execute program code for database query module 104, where database query module 104 can cause processor 106 to query health context database 112 to extract corresponding health context data entries based on the audience file. Health context data entries may include at least one field for a social determinant of health, such as non-medical factors that can influence health outcomes, including socioeconomic status, education, and/or occupation. The health context data entries can be organized based on key points of intersection such as a geographic location (e.g., a zip code) and demographic characteristics (e.g., age, gender). Other fields that may be captured in health context data entries may include a marital status, a household income, an education level, an ethnicity, an occupation, a family structure, and a homeownership status. Processor 106 may determine that the corresponding health context data entries include an age field with an age value that is within the age range of the audience definition and that the corresponding health context data entries include a field closely related to a medical condition, where the value is closely related to the medical condition of the audience definition. In some embodiments, processor 106 may determine that the corresponding health context data entries include a geographic location field with a geographic location value that matches or closely matches a geographic location found in the audience definition and that the corresponding health context data entries include the geographic location field. Similarly, processor 106 may determine the corresponding health context data entries include a gender field matching the gender in the audience definition. In this way, processor 106 and database query module 104 may precisely identify and pinpoint health context data entries that may be directly relevant to and/or match the audience definition.

The program code for the database query module, when executed by the processor, can cause the processor to query an online activity database to extract corresponding online activity data entries based on the audience file. For example, processor 106 may execute program code for database query module 104, wherein database query module 104 may cause processor 106 to query online activity database 114 to extract corresponding online activity data entries based on the audience file. Online activity data entries may include data entries including at least one field associated with online activity of a user, such data collected from web activity using tracking pixels, which may be embedded on client websites. These tracking pixels may gather anonymized data about the demographic and/or social aspects of a user of a website, providing information about the online habits and interests of the health audience defined by the audience definition. Processor 106 may determine that the corresponding online activity data entries include an age field with an age value that is within the age range of the audience definition and that the corresponding online activity data entries include an online activity field for a particular website or online application, where the online activity field corresponds to an anonymous user, where the age field includes an age value that matches or closely matches the age range of the audience definition. In this way, processor 106 and database query module 104 may precisely identify and pinpoint online activity data entries that may be directly relevant to and/or match the audience definition.

The program code for the database query module, when executed by the processor, can cause the processor to synthesize the extracted corresponding health data entries, corresponding health context data entries, and corresponding online activity data entries to generate de-identified aggregate data in an aggregate data file. For example, processor 106 may execute program code for database query module 104, wherein database query module 104 may cause processor 106 to synthesize the extracted corresponding health data entries, corresponding health context data entries, and corresponding online activity data entries. In some embodiments, processor 106 can receive the corresponding health data entries, the corresponding health context data entries, and the corresponding online activity data entries without PII to generate de-identified aggregate data in an aggregate data file, where PII is not present in any of the corresponding data entries. As used herein, PII may include any data or information of a user that could identify the user, such as name, social security number, address, and/or the like. Generally, data or information such as gender, geographic location (e.g., zip code), or age is not considered to be PII.

The program code for the modeling module, when executed by the processor, can cause the processor to clean the de-identified aggregate data to generate consistent data fields within the aggregate data file. For example, processor 106 may execute program code for modeling module 116, where modeling module 116 can cause processor 106 to clean the de-identified aggregate data to generate consistent data fields within the aggregate data file. In this way, the aggregate data file can have consistent data entries with no missing fields such that the aggregate data file may be analyzed, for example, by one or more machine learning models. The aggregate data field having consistent data entries can also allow for more accurate results for scoring values that can be appended to target data entries in a target database. The more accurate the scoring values are by having complete and consistent aggregated data, the more relevant and accurate the annotated target data entries can be for analysis and use for targeting specific client devices.

The program code for the modeling module, when executed by the processor, can cause the processor to automatically cluster the de-identified aggregate data into plural segments based on at least one of an age value, a geographic location value, and/or a gender value. For example, processor 106 may execute program code for modeling module 116, where modeling module 116 can cause processor 106 to automatically cluster the de-identified aggregate data into plural segments based on an age value, a geographic location value, and/or a gender value. A segment may include aggregated data based at least on age, geographic location, and/or gender. For example, the segment may include a gender, an age, a geographic location, and a count of a number of users having age values, geographic location values, and/or gender values that are equal and/or the same. Within the aggregate data file, if there are six data entries for males of age 34 that were collected from the database, then a segment may be generated including data files and values of age: 34, gender: M, and count: 6. Other segments (e.g., based on geographic location and/or gender) may be generated in a similar manner based on the aggregate data file.

The program code for the modeling module, when executed by the processor, can cause the processor to calculate plural joint probability values based on the plural segments, and at least the age ranges and age values, geographic location values, and/or gender values defined by aggregated audience data objects. For example, processor 106 may execute program code for modeling module 116, wherein modeling module 116 may cause processor 106 to calculate plural joint probability values based on the plural segments, and at least the age ranges and age values, geographic location values, and/or gender values defined by aggregated audience data objects. The joint probability values can be calculated based on values such as a claims probability, a patient probability, a social determinant of health probability, and/or an online probability, where each of these probabilities may be generated based on their respective data entries in the aggregate data file.

The program code for the modeling module, when executed by the processor, can cause the processor to calculate a final probability score for each of the plural segments using the plural joint probability values and weights assigned to each field in the audience data objects to generate an indexing model. For example, processor 106 may execute program code for modeling module 116, where modeling module 116 can cause processor 106 to calculate a final probability score for each of the plural segments using the plural joint probability values and weights assigned to each field in the audience data objects to generate an indexing model. The calculated probabilities can be combined with demographic data to compute a final probability score for each segment. The final probability score may be weighted according to importance of different factors such as age and/or claim counts, resulting in a more accurate representation of the likelihood of specific outcomes.

The program code for the modeling module, when executed by the processor, can cause the processor to query a target database, including data entries with at least an age field, a gender field, and/or a geographic location field. For example, processor 106 may execute program code for modeling module 116, wherein modeling module 116 may cause processor 106 to query a target database, including data entries with at least an age field, a gender field, and a geographic location field. Modeling module 116 may cause processor 106 to request data entries from the target database which have closely matching age values, gender values, and/or geographic location values. In some embodiments, target database 118 can include a third-party database (e.g., owner and/or controlled by a third party separate from computing system 100 and/or health database annotation system 402. Each target data entry in the target database can be associated with a data publisher identifier. The data publisher identifier can be associated with a client device (e.g., a computing device used by a user, a television spot, and/or the like).

The program code for the modeling module, when executed by the processor, can cause the processor to append each of the final probability scores calculated for each of the plural segments to a target data entry in the target database, wherein at least one value in the segment matches with the age field, the gender field, and/or the geographic location field from the target data entry in the target database. For example, processor 106 may execute program code for modeling module 116, where modeling module 116 can cause processor 106 to append each of the final probability scores calculated for each of the plural segments to a target data entry in target database 118, where at least one value in the segment matches with the age field, the gender field, and/or the geographic location field from the target data entry in target database 118.

In some embodiments, the target database can include a third-party database and each target data entry in the target database can be associated with a data publisher identifier (ID). For example, target database 118 can include a third-party database where each target data entry in target database 118 can include a data publisher ID field. The data publisher ID field can include a data publisher ID that identifies a client device and/or a data publisher. In some embodiments, the data publisher ID may be associated with a client device through other means of identification (e.g., if the client device is associated with an account/user account for an application provided by the data publisher, etc.).

The program code for the modeling module, when executed by the processor, can cause the processor to transmit updated health information to one or more client devices associated with at least one segment such that the updated health information is transmitted to users where the updated health information is useful to the client devices without being associated to any PII of the users. For example, processor 106 may execute program code for modeling module 116, wherein modeling module 116 may cause processor 106 to transmit updated health information to one or more client devices associated with at least one segment such that the updated health information is transmitted to users where the updated health information is useful to the client devices without being associated to any PII of the users. Updated health information, as used herein, may include information relating to one or more health products or one or more health services.

As shown in FIG. 1, computing system 100 can include audience definition module 102, database query module 104, processor 106, memory 108, health data database 110, health context database 112, online activity database 114, modeling module 116, and target database 118. Computing system 100 can include at least one computing device connected to a network. In some embodiments, computing system 100 can include components shown in FIG. 1 in a single computing device or computing system. Alternatively, computing system 100 can include components shown in FIG. 1 distributed across multiple computing devices and/or computing systems.

Computing system 100 can include audience definition module 102. Audience definition module 102 can include program code for managing data aggregations and annotating target databases. Specifically, audience definition module can include program code for receiving text input data and converting the text input data into audience definition data, including one or more parameters to define a collection of users. The one or more parameters may include age, geographic location, gender, income, occupation, or other factors associated with a user. Audience definition module 102 can be executed by processor 106 to receive text input data and to generate audience definition data based on the text input data. Audience definition module 102 may transmit the audience definition data among applications and/or modules operating within computing system 100.

Computing system 100 can include database query module 104. In some embodiments, database query module 104 can include program code for querying various databases and comparing audience definition data with data entries stored in the various databases. Database query module 104 can also include program code for retrieving data entries from the various databases.

Computing system 100 can include processor 106 (e.g., a specially configured processor, CPU, and/or the like) and memory 108. Processor 106 can execute software instructions (e.g., compiled program code) for audience definition module 102, database query module 104, and modeling module 116.

Computing system 100 can include one or more computing devices including one or more processors (e.g., processor 106) configured to execute software instructions. For example, computing system 100 can include a desktop computer, a portable computer (e.g., laptop computer, tablet computer), a workstation, a mobile device (e.g., smartphone, cellular phone, personal digital assistant, wearable device), a server, and/or other like devices. Computing system 100 can include a computing device configured to communicate with one or more other computing devices over a network. Computing system 100 can include a group of computing devices (e.g., a group of servers) and/or other like devices. In some embodiments, computing system 100 can include one or more data storage devices (e.g., health data database 110, health context database 112, and/or online activity database 114). Alternatively, a data storage device can be separate from computing system 100 and can be in communication with computing system 100 over a communication network.

Processor 106 can be implemented in hardware, software, or a combination of hardware and software. For example, processor 106 can include a common processor (e.g., a CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed with software instructions and/or can execute software instructions to perform a function. Processor 106 can be coupled to memory 108 via a data bus to transfer data between processor 106 and memory 108.

Memory 108 can include random access memory (RAM), read-only memory (ROM), and/or another type of dynamic or static storage device (e.g., flash memory, magnetic memory, optical memory, etc.) that stores information and/or software instructions for use by processor 106. Memory 108 can include a computer-readable medium and/or storage component. A computer-readable medium (e.g., a non-transitory computer-readable medium) is defined herein as a non-transitory memory device. A non-transitory memory device includes memory space located inside of a single physical storage device or memory space spread across multiple physical storage devices. In some embodiments, memory 108 can include one or more storage locations for storing data and/or data entries, such as health data entries and/or audience definition data.

Software instructions can be read into memory 108 from another computer-readable medium or from another device via a communication interface with computing system 100. When executed, software instructions stored in memory 108 can cause processor 106 to perform one or more processes and/or functions described herein. Embodiments described herein are not limited to any specific combination of hardware circuitry and software and can include various combinations of hardware circuitry and software.

Health data database 110, health context database 112, and/or online activity database 114 each can include random access memory (RAM), read only memory (ROM), and/or another type of dynamic or static storage device (e.g., flash memory, magnetic memory, optical memory, etc.) that stores information for use by computing system 100 and/or processor 106. For example, health data database 110 can store health data entries or other data used by audience definition module 102, database query module 104, and/or modeling module 116. Similarly, health context database 112 may store health context data entries and online activity database 114 may store online activity data entries, where each of the health context data entries and the online activity data entries can be used by audience definition module 102, database query module 104, and/or modeling module 116 to perform functions described herein. In some embodiments, health data database 110, health context database 112, and/or online activity database 114 each can include a non-transitory computer readable medium that can store information, software, and/or machine learning models related to the operation and use of computing system 100, audience definition module 102, database query module 104, modeling module 116, and/or processor 106. For example, health data database 110, health context database 112, and/or online activity database 114 each can include a hard disk (e.g., a magnetic disk, an optical disk, a magneto-optic disk, a solid-state disk, etc.) and/or another type of computer-readable medium.

In some embodiments, the health data database can contain information concerning medical claims, treatment histories, prescription information, and other healthcare information. For example, health data database 110 can store medical claim data in a medical claim field, where the medical claim field is associated with an age field, a geographic location field, and/or a gender field in a health data entry.

In some embodiments, the health context database can contain information concerning socioeconomic status, education, occupation, marital status, household income, education level, ethnicity, occupation, family structure, and/or homeownership status. For example, health context database 112 can store health context data in at least one health context field (e.g., an education field, a household income field, an occupation field, and/or the like) associated with an age field, a geographic location field, and/or a gender field in a health context data entry.

In some embodiments, health data database 110, health context database 112, and/or online activity database 114 each can include a computing device (e.g., a database device) configured to communicate with processor 106 (e.g., via one or more applications and/or modules such as audience definition module 102, database query module 104, and/or modeling module 116) via a bus or a network environment. For example, health data database 110, health context database 112, and/or online activity database 114 each can include a server, a group of servers, and/or other like devices. In some embodiments, health data database 110, health context database 112, and/or online activity database 114 each can be associated with one or more computing devices providing interfaces such that a user and/or an application and/or module can interact with health data database 110, health context database 112, and/or online activity database 114 each via the one or more computing devices. Health data database 110, health context database 112, and/or online activity database 114 each can be in communication with computing system 100 and/or processor 106 such that each of health data database 110, health context database 112, and/or online activity database 114 is separate from computing system 100 and/or processor 106. Alternatively, each of health data database 110, health context database 112, and/or online activity database 114 can be part of (e.g., a component of) computing system 100 (e.g., as shown in FIG. 1).

In some embodiments, health data database 110, health context database 112, and/or online activity database 114 each can include a device capable of storing data (e.g., a data storage device). In some embodiments, health data database 110, health context database 112, and/or online activity database 114 each can include a collection of data (e.g., data elements, data entries, etc.) stored and accessed by one or more computing devices, applications, and/or modules. Health data database 110, health context database 112, and/or online activity database 114 each can include file system storage, cloud storage, in-memory storage, and/or the like. Health data database 110, health context database 112, and/or online activity database 114 each can include non-volatile storage (e.g., flash memory, magnetic media), volatile storage (e.g., random access memory (RAM)), or both non-volatile and volatile storage. In some embodiments, health data database 110, health context database 112, and/or online activity database 114 each can be hosted (e.g., stored and permitted to be accessed by other computing devices via a network environment) on a computing device separate from computing system 100. Health data database 110, health context database 112, and/or online activity database 114 each can be configured to communicate with processor 106 via one or more applications and/or application modules, such as audience definition module 102, database query module 104, and/or modeling module 116.

As used herein, a module (e.g., software module, software/hardware module, and/or the like), a service (e.g., software service, microservice, and/or the like), or an application can refer to a loosely-coupled software application and/or a loosely-coupled software service that is designed to facilitate software reuse and high cohesion. In a microservice architecture, software services are fine-grained and include protocols that are generally lightweight. Software modules and/or services can include interfaces which are treated as a public application programming interface (API). The software module and/or software service can exist and may be reusable (e.g., portable to other software applications and/or systems without requiring changes to the module) independent of other software modules and/or software services.

As disclosed herein, a module can include software, hardware, or a combination of software and hardware. As an example, where audience definition module 102 includes a software module, audience definition module 102 can be configured as program code to cause processor 106 to perform various functions. Alternatively, where audience definition module 102 includes software and hardware, audience definition module 102 can be configured as program code combined with hardware (e.g., a specially configured processor, an embedded system, and/or the like) to perform various functions independent of and/or in conjunction with processor 106. In this way, audience definition module 102 (and other modules disclosed herein) can be configured with its own hardware and/or processor for performing various functions and audience definition module 102 can be integrated with computing system 100 and/or processor 106. It should be understood that, although this example is described with respect to audience definition module 102, other modules disclosed herein (database query module 104, modeling module 116) may be programmed or configured with similar properties and/or functions.

Audience definition module 102 can include a software module (e.g., a module invoked by processor 106 based on program code executed by processor 106) such that functionalities of audience definition module 102 can be accessed via an API. In some embodiments, audience definition module 102 can include a software module such that audience definition module 102 can be packaged into a single unit (e.g., a single unit of reusable program code) that can be easily deployed and/or shared. In some embodiments, audience definition module 102 can include a combination of hardware and software (e.g., a specially configured processor to perform certain functions) such that audience definition module 102 can perform functions and share data and/or commands with processor 106. Audience definition module 102 can include various functions (e.g., via hardware or software) that can cause processor 106 to manipulate objects and/or data (e.g., data entries, audience definition data, text input data) to manage data aggregations and/or annotate target databases.

As an example, audience definition module 102 can be configured to receive text input defining an audience definition. In some embodiments, the text input can include at least an age range and a medical condition. An age range could include a range of values representing ages, such as ages 18-35, or some other range of age values. A medical condition can include a value or text data representing a medical condition. For example, a medical condition could include text data such as “diabetes” indicating a medical condition of diabetes. In some other embodiments, a medical condition could be represented by a Boolean value combined with a label, indicating that a data entry includes a “True” (1) or “False” (0) for a property of a medical condition, such as diabetes. Audience definition module 102 can be configured to structure the text input into an audience data object in an audience file including at least an age range field and a medical condition field.

In some embodiments, the audience data object can include an insurance provider field and a geographic location field. Insurance provider data stored in the insurance provider field in the audience data object can be replaced with an insurance provider code. Geographic location data stored in the geographic location field in the audience data object can be replaced with a geographic location code.

Database query module 104 can include a software module (e.g., a module invoked by processor 106 based on program code executed by processor 106) such that functionalities of database query module 104 can be accessed via an API. In some embodiments, database query module 104 can include a software module such that database query module 104 can be packaged into a single unit (e.g., a single unit of reusable program code) that can be easily deployed and/or shared. In some embodiments, database query module 104 can include a combination of hardware and software (e.g., a specially configured processor to perform certain functions) such that database query module 104 can perform functions and share data and/or commands with processor 106. Database query module 104 can include various functions (e.g., via hardware or software) that can cause processor 106 to manipulate objects and/or data (e.g., data entries, audience definition data, text input data) to manage data aggregations and/or annotate target databases.

As an example, database query module 104 can be configured to query a health data database to extract corresponding health data entries based on the audience file. In some embodiments, database query module 104 can be configured to query a health context database to extract corresponding health context data entries based on the audience file. In some embodiments, database query module 104 can be configured to query an online activity database to extract corresponding online activity data entries based on the audience file. In some embodiments, database query module 104 can be configured to synthesize the extracted corresponding health data entries, corresponding health context data entries, and corresponding online activity data entries, the corresponding health context data entries, and the corresponding online activity data entries being devoid of PII (e.g., not including any PII) to generate de-identified aggregate data in an aggregate data file. In this way, the synthesized data in the aggregate data file does not include any PII and can be used for anonymized analysis.

Modeling module 116 can include a software module (e.g., a module invoked by processor 106 based on program code executed by processor 106) such that functionalities of modeling module 116 can be accessed via an API. In some embodiments, modeling module 116 can include a software module such that modeling module 116 can be packaged into a single unit (e.g., a single unit of reusable program code) that can be easily deployed and/or shared. In some embodiments, modeling module 116 can include a combination of hardware and software (e.g., a specially configured processor to perform certain functions) such that modeling module 116 can perform functions and share data and/or commands with processor 106. Modeling module 116 can include various functions (e.g., via hardware or software) that can cause processor 106 to manipulate objects and/or data (e.g., data entries, audience definition data, text input data) to manage data aggregations and/or annotate target databases.

As an example, modeling module 116 can be configured to clean the de-identified aggregate data to generate consistent data fields within the aggregate data file. As part of cleaning the de-identified aggregate data, modeling module may fill in fields that have missing data entries, or modeling module may change existing data entries that are different from a majority of data entries to make all data entries in a particular field consistent. For example, a majority of data entries may include the character “M” in the gender data field. However, some data entries may include “m” in the gender data field. Modeling module may make the gender data field consistent across all data entries by changing the “m” values to “M” to match the majority of existing values in the gender data field for the data entries. Other operations may be performed by modeling module 116 as part of cleaning the de-identified aggregate data.

In some embodiments, modeling module 116 may be configured to automatically cluster the de-identified aggregate data into plural segments based on an age value. For example, modeling module may cluster the de-identified data based on whether the age value in an age field of each data entry falls within an age range, such as ages 30-40, or another range. In this way, modeling module may divide and segment the data based on the age field in the data entries, while still maintaining relevant groups of aggregate data.

In some embodiments, modeling module 116 may calculate plural joint probability values based on the plural segments, and at least the age ranges and the medical conditions defined by aggregated audience data objects. Joint probability values may be combined probabilities based on one or more of a claim probability, a patient probability, a social determinant of health probability, and/or an online probability.

In some embodiments, modeling module 116 can be configured to calculate a final probability score for each of the plural segments using the plural joint probability values and using weights assigned to each field in the audience data objects to generate an indexing model. Weights may be assigned to each field in an audience data object based on each segment (e.g., how the segments are generated and based on properties such as age range). Some fields may be assigned higher or lower weights depending on how much the data field is determined to affect an outcome of the analysis.

In some embodiments, modeling module 116 can be configured to query a target database, including data entries with at least an age field, a gender field, and a geographic location field. For example, modeling module 116 may query target database 118 to find data entries including similar values in various fields of the data entries, where the values include de-identified data. As shown in FIG. 1, such fields may include age, gender, and/or geographic location (e.g., zip code). If at least some of the fields and/or values match, modeling module 116 may identify and/or retrieve the matching data entries from target database 118 for scoring.

In some embodiments, modeling module 116 may be configured to append each of the final probability scores calculated for each of the plural segments to a target data entry in the target database, where the segment matches with the age field, the gender field, and the geographic information field from the target data entry in the target database. As shown in FIG. 1, the final probability score can be appended to each data entry as a new data field.

In some embodiments, the target database can include individual entries for a unique user. For example, target database 118 can include individual data entries, where each individual entry corresponds to a unique user. Alternatively, target database 118 can include data entries which correspond to a client device, or a location (e.g., a single device within a home, a location of a home, etc.). The target database can include individual entries for a unique television spot defined at least by a time slot and an audience size. For example, target database 118 can include individual entries where each individual entry represents a unique television spot defined at least by a time slot and an audience size. In some embodiments, the target database can include individual entries for an out of home placement defined at least by a physical location, an audience size, and a demographic reach. For example, target database 118 can include individual data entries, each data entry representing an out of home placement defined at least by a physical location, an audience size, and a demographic reach (e.g., the individual data entry including a physical location field, an audience size field, and a demographic reach field). In some embodiments, the individual entries for an out of home placement can be selected from at least one of a billboard advertisement along a roadway, an advertisement in a shopping mall, an advertisement in a public transit system, and/or an advertisement at a sporting venue.

In some embodiments, modeling module 116 may transmit updated health information to one or more client devices associated with at least one segment such that the updated health information is transmitted to users where the updated health information is useful to the client devices without being associated to any PII of the users. That is, updated health information, such as health products and/or health services, may be transmitted to client devices based on the final probability score and the age, gender, and/or geographic location of a user. This information of the user may be associated with a client device via a publisher ID, where the publisher ID may be associated with a client device based on an application and/or service operating on the client device. In some instances, the publisher ID may be based on an owner of the client device.

Target database 118 can include random access memory (RAM), read only memory (ROM), and/or another type of dynamic or static storage device (e.g., flash memory, magnetic memory, optical memory, etc.) that stores information for use by computing system 100 and/or processor 106. For example, target database 118 can store health data entries or other data used by audience definition module 102, database query module 104, and/or modeling module 116. In some embodiments, target database 118 can include a non-transitory computer readable medium that can store information, software, and/or machine learning models related to the operation and use of computing system 100, audience definition module 102, database query module 104, modeling module 116, and/or processor 106. For example, target database 118 can include a hard disk (e.g., a magnetic disk, an optical disk, a magneto-optic disk, a solid-state disk, etc.) and/or another type of computer-readable medium.

In some embodiments, target database 118 can include a computing device (e.g., a database device) configured to communicate with processor 106 (e.g., via one or more applications and/or modules such as audience definition module 102, database query module 104, and/or modeling module 116) via a bus or a network environment. For example, target database 118 can include a server, a group of servers, and/or other like devices. In some embodiments, target database 118 can be associated with one or more computing devices providing interfaces such that a user and/or an application and/or module can interact with target database 118 via the one or more computing devices. Target database 118 can be in communication with computing system 100 and/or processor 106 such that target database 118 is separate from computing system 100 and/or processor 106. Alternatively, target database 118 can be part of (e.g., a component of) computing system 100 (e.g., as shown in FIG. 1).

In some embodiments, target database 118 can include a device capable of storing data (e.g., a data storage device). In some embodiments, target database 118 can include a collection of data (e.g., data elements, data entries, etc.) stored and accessed by one or more computing devices, applications, and/or modules. Target database 118 can include file system storage, cloud storage, in-memory storage, and/or the like. Target database 118 can include non-volatile storage (e.g., flash memory, magnetic media), volatile storage (e.g., random access memory (RAM)), or both non-volatile and volatile storage. In some embodiments, target database 118 can be hosted (e.g., stored and permitted to be accessed by other computing devices via a network environment) on a computing device separate from computing system 100. Target database 118 can be configured to communicate with processor 106 via one or more applications and/or application modules, such as audience definition module 102, database query module 104, and/or modeling module 116.

The number and arrangement of systems, hardware, and/or modules shown in FIG. 1 is provided as an example. There can be additional systems, hardware, and/or modules, fewer systems, hardware, and/or modules, different systems, hardware, and/or modules, or differently arranged systems, hardware, and/or modules than those shown in FIG. 1. Furthermore, two or more systems, hardware, and/or modules shown in FIG. 1 can be implemented within a single system, hardware, and/or module. A single system, hardware, and/or module shown in FIG. 1 can be implemented as multiple, distributed systems, hardware, and/or modules. Additionally, or alternatively, a set of systems, a set of hardware, and/or a set of modules (e.g., one or more systems, one or more hardware devices, one or more modules) of FIG. 1 can perform one or more functions described as being performed by another set of systems, another set of hardware, or another set of modules of FIG. 1.

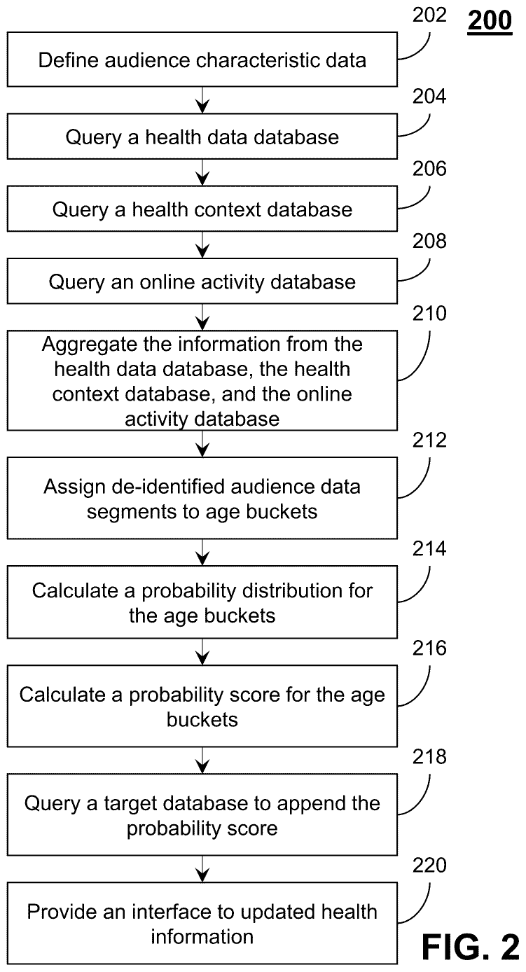

FIG. 2 shows a flow diagram of an exemplary method for managing data aggregations and/or annotating target databases as disclosed herein. In some embodiments, one or more of the functions described with respect to method 200 can be performed (e.g., completely, partially, etc.) by computing system 100 and/or any one or multiple of audience definition module 102, database query module 104, and/or modeling module 116 (e.g., via processor 106). In some embodiments, one or more of the steps of method 200 can be performed (e.g., completely, partially, etc.) by another system, hardware, or module or a group of systems, hardware, or modules separate from or including computing system 100 and/or audience definition module 102, database query module 104, and/or modeling module 116, such as a client device, a separate computing device, or an additional application and/or module.

As shown in FIG. 2, at step 202, method 200 can include defining audience characteristic data. For example, audience definition module 102 (e.g., executed by processor 106) can define audience characteristic data. The audience characteristic data may include at least an age range and a medical condition. Audience definition module 102 may define the audience characteristic data using a range of age values (e.g., 20-30) and a value for a medical condition (e.g., in text form, Boolean, or other data type to denote a medical condition). In some embodiments, audience definition module 102 may define the audience characteristic data based on text input from a user using an input device. In some embodiments, the audience characteristic data can include at least one of a set of geographic regions, a prescribed treatment, and/or insurance coverage information.

At step 204, method 200 can include querying a health data database. For example, database query module 104 can query health data database 110 including health data entries. Each health data entry may include at least an age field, a gender field, and/or a geographic location field. The audience characteristic data can be received as health information including a set of individual data points (e.g., a data field) paired with an age value, a gender character, and a geographic location value which is correlated with the health information.

In some embodiments, the querying of the health data database can include limiting the received health information to entries corresponding to the medical condition of the audience characteristic data. For example, processor 106 can query health data database 110 to limit the received health information to data entries corresponding to the medical condition of the audience characteristic data.

At step 206, method 200 can include querying a health context database. For example, database query module 104 can query a health context database including health context data entries, each health context data entry including at least an age field, a gender field, and a geographic location field. The audience characteristic data can be received as health context information including a set of individual data points paired with an age value, a gender value (e.g., a character), and a geographic location value which is correlated with the health context information. In some embodiments, health context information may include data related to factors that may be relevant to health outcomes, such as income, occupation, and other factors including age and/or geographic location.

At step 208, method 200 can include querying an online activity database. For example, database query module 104 can query an online activity database including online activity data entries. Each online activity data entry may include at least an age, a gender, and a geographic location field, wherein the audience characteristic data is received as online activity information including a set of individual data points paired with an age value, a gender value (e.g., a character), and a geographic location value which is correlated with the online activity information. In some embodiments, online activity data entries may include data related to online activity of a user, such as data from tracking cookies, or data collected by other applications related to a user's online activity.

At step 210, method 200 can include aggregating the information from the health data database, the health context database, and the online activity database. For example, database query module 104 (e.g., when executed by processor 106) can aggregate the health information received from the health data database, the health context information received from the health context database, and the online activity information received from the online activity database into a set of de-identified audience data segments organized by age, gender, and geographic location. Database query module 104 may aggregate all of the collected data into a single file and/or database. The set of de-identified audience data segments can be devoid of PII, which may mean that the set of de-identified audience data segments does not include any PII in any data fields or data entries. In some embodiments, each de-identified audience data segment in the set may include at least a count for the segment and values for the individual data points. For example, a count may be a value representing a number of data entries which include a matching age value, a matching gender value, and/or a matching geographic location value within a segment.

At step 212, method 200 can include assigning de-identified audience data segments to age buckets. For example, modeling module 116 can assign each of the de-identified audience data segments of the set to an age bucket within a set of age buckets. Each of the age buckets can be defined by an age range and a gender. For example, a first age bucket may include a gender of male (“M”) and an age range of 21-30, while a second age bucket may include a gender of male and an age range of 31-40, while a third age bucket may include a gender of female (“F”) and an age range of 31-40, etc. It should be understood that there is no prescribed limit to the number and types of age buckets that may be defined for the de-identified audience data segments.

In some embodiments, assigning each of the de-identified audience data segments of the set to an age bucket within a set of age buckets can be performed dynamically using a clustering technique. For example, processor 106 can use the clustering technique to generate the set of age buckets and separate each of the age buckets by age to identify optimal split points based on counts in the de-identified audience data segments.

At step 214, method 200 can include calculating a probability distribution for the age buckets. For example, modeling module 116 can calculate a probability distribution for the set of age buckets based on probabilities such as a claim probability, a patient probability, a social determinant of health probability, and/or an online activity probability.

At step 216, method 200 can include calculating a probability score for the age buckets. For example, modeling module 116 can calculate a probability score for each of the age buckets in the set using the probability distribution and weights assigned to the individual data points. Weight may be assigned to the individual data points based on various factors to weight different properties, such as age, where those properties may have more effect on health data.

In some embodiments, calculation of the probability score for each of the age buckets can include initially normalizing the probability scores for each of the age buckets using a square root transformation to apply a mathematical spread to highlight differences between the age buckets. Subsequently, calculation of the probability scores can include scaling the normalized probability scores for each of the age buckets to arrive at probability values between 51 and 100.

At step 218, method 200 can include querying a target database to append the probability score. For example, modeling module 116 can query target database 118. Target database 118 may be keyed by at least age, gender, and/or geographic location. In some embodiments, target database 118 may include a target database including one or more data entries including fields for health related information. In some embodiments, target database 118 can include other types of databases storing other types of information associated with at least an age field, a gender field, and/or a geographic location field. Modeling module 116 can append the calculated probability score of the age bucket to one or more target data entries in the target database that matches with the age, gender, and geographic information from the target database. In this way, modeling module 116 may score target data entries based on the de-identified aggregate, thus providing health information to relevant segments of an audience based on the analysis of data in health data database 110, health context database 112, and/or online activity database 114.

In some embodiments, processor 106 can assign each of the de-identified audience data segments of the set to any one or more of a claim count bucket within a set of claim count buckets, a gender bucket within a set of gender buckets, a social determinant of health bucket within a set of social determinants of health buckets, and a patient bucket within a set of patient buckets, the claim count buckets defined by a claim count range and a gender; the gender bucket defined by a gender identifier, the social determinant of health bucket defined by a context attribute, and the patient bucket defined by a range of patient counts. Processor 106 can calculate a probability distribution for any one or more of the set of claim count buckets, the set of gender buckets, the set of social determinants of health buckets, and the set of patient buckets. Processor 106 can also calculate a probability score for each bucket in each set using the probability distribution and weights assigned to the individual data points.

At step 220, method 200 can include providing an interface to updated health information without PII to the target database. For example, modeling module 116 (or another module, via processor 106) can provide an interface to updated health information for target database 118 (and/or a computing device owning and/or controlling target database 118) for transmitting the updated health information to one or more client devices associated with at least one audience data segment of the set of de-identified audience data segments such that the updated health information is targeted based on relevance to users of the client devices without being associated to any PII. In this way, users may receive the updated health information via a client device they are associated with (e.g., a client device used by a user) and the user does not have to be concerned with any PII being used or shared among organizations or devices. Users can be confident that they receive updated, relevant health information while their PII and identities are protected. Target database 118 can request the updated health information from, for example, modeling module 116 for transmitting the updated health information to the one or more client devices.

The method 200, further comprising transmitting the updated health information to one or more client devices associated with at least one audience data segment of the set of de-identified audience data segments such that the updated health information is targeted based on relevance to users of the one or more client devices without being associated to any PII.

In some embodiments, modeling module 116 can respond to a request from target database 118, where the request was transmitted by target database 118 and/or another device associated with target database 118. The request can request the updated health information associated with a specific data entry and probability score stored in target database 118. Modeling module 116 can then transmit updated health information to target database 118 for transmitting the updated health information to one or more client devices.

Steps of method 200 can be performed in various orders and sequences and are not necessarily limited to being performed in the order shown in FIG. 2. Accordingly, steps of method 200 are not limited to any particular order and can be performed by various components, whether computing system 100 is implemented on a single computing device or multiple, distributed computing devices. Steps of method 200 can also be performed by a single processor of computing system 100 or by multiple processors of computing system 100.

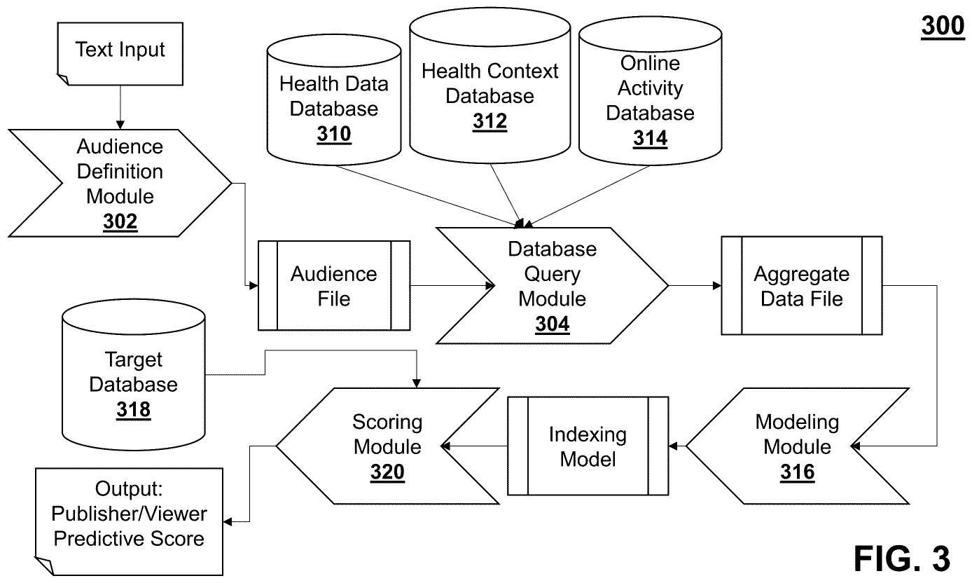

FIG. 3 shows a flow diagram of an exemplary system and/or components pipeline 300 for generating and assessing data aggregations and/or annotating target databases disclosed herein. As shown in FIG. 3, system and/or components pipeline 300 may include audience definition module 302, an audience file, database query module 304, an aggregate data file, health data database 310, health context database 312, online activity database 314, modeling module 316, an indexing model, target database 318, scoring module 320, and associated output. Audience definition module 302 may be the same as or similar to audience definition module 102. Database query module 304 may be the same as or similar to database query module 104. Health data database 310, health context database 312, and online activity database 314 may be the same as or similar to health data database 110, health context database 112, and online activity database 114, respectively. Modeling module 316 may be the same as or similar to modeling module 116. Target database 318 may be the same as or similar to target database 118.

As shown in FIG. 3, system and/or components pipeline 300 may being by receiving text input at audience definition module 302 (e.g., from a user via an input device). Audience definition module 302 may use the text input to generate an audience file including audience data objects. Each audience data object may include audience data fields, including at least an age field, a gender field, and/or a geographic location field. Each of the audience data fields defined in the audience data objects may be generated and defined based on the text input to audience definition module 302.

The audience files may be input to database query module 304 to define the audience data objects and audience data fields that database query module 304 should look for in various databases storing user and/or patient data. Database query module 304 may use the audience data file (including audience data objects with audience data fields) to query various databases such as health data database 310, health context database 312, and/or online activity database 314 to retrieve data relating to and/or matching the audience data objects and audience data fields. The retrieved data may include additional information, such as health information, health context information, and/or online activity information associated with the data fields.

From the retrieved data, database query module 304 may generate an aggregate data file compiling and/or synthesizing all of the retrieved data, in formats closely matching the audience definition data objects, such that the compiled data includes audience definition data (e.g., gender, age, geographic location), health information, health context information, and/or online activity information in compiled data entries in the aggregate data file.

The aggregate data file may then be used as input to modeling module 316 to model the aggregate data (e.g., without any PII present in the aggregate data file). In this way, the aggregate data file does not include any PII and thus cannot result in any security issues or data breaches relating to PII. Modeling module 316 may then generate an indexing model. The indexing model can be an internal model representation that consolidates the aggregate data file, any calculated probabilities, and additional demographic data produced by modeling module 316. The indexing model may act as a bridge between modeling module 316 and scoring module 320, providing a unified structure for scoring against third-party databases, such as target database 318. In some embodiments, the indexing model may or may not be stored, either in system 300 or another location. The indexing model logically separates preparation of audience segments from scoring performed by scoring module 320. In this way, system and/or components pipeline 300 may ensure that data in the aggregate data file is ready to be applied to different datasets in different third-party database, such as target database 118.

The indexing model and associated data in the aggregate data file is transmitted to scoring module 320 for probability calculations and final probability scoring. Once final probability score is calculated for each data entry, scoring module 320 can output a publisher/viewer predictive score to append to data entries in third-party databases, such as target database 118. Examples of publisher/viewers may include a television and/or radio spot, an out of home (OOH) placement, a connected television household, a website user, and/or the like.

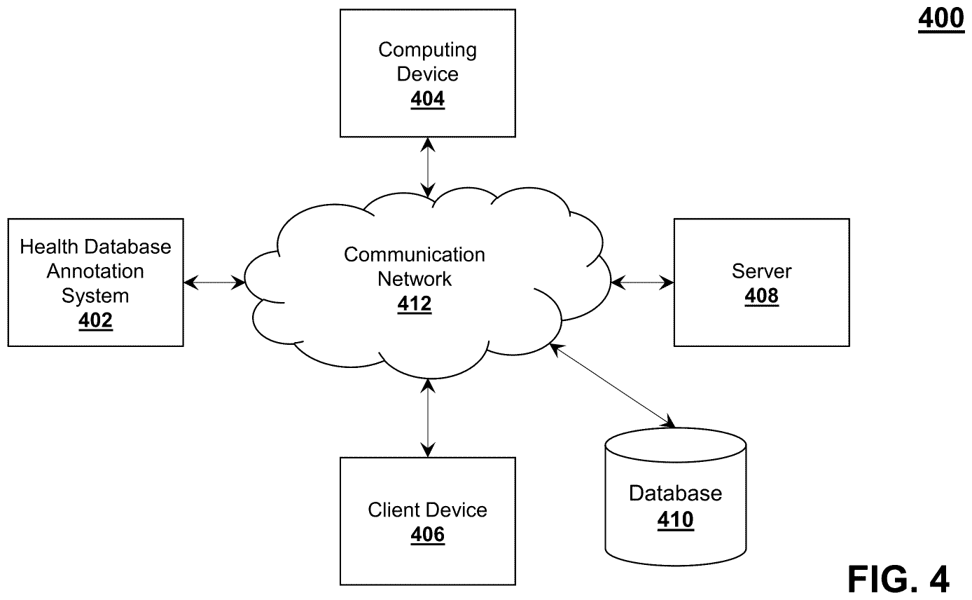

Referring to FIG. 4, shown is a diagram of an exemplary environment 400 in which methods, systems, and/or computer program products, described herein, may be implemented as disclosed herein. As shown in FIG. 4, environment 400 may include health database annotation system 402, computing device 404, client device 406, server 408, database 410, and communication network 412. In some embodiments, each of computing device 404, client device 406, server 408, database 410, and/or communication network 412 may be implemented by (e.g., as part of) health database annotation system 402. In some embodiments, at least one of each of computing device 404, client device 406, server 408, database 410, and/or communication network 412 may be implemented by (e.g., as part of) another system, another device, another group of systems, or another group of devices, separate from or including health database annotation system 402, such as computing device 404, client device 406, server 408, database 410, and/or the like.

Health database annotation system 402 may include one or more devices capable of receiving information from and/or communicating information to computing device 404, client devices 406, server 408, and/or database 410 via communication network 412. For example, health database annotation system 402 may include a computing device, such as a server, a group of servers, and/or other like devices. In some embodiments, health database annotation system 402 may be associated with a server as described herein. In some embodiments, health database annotation system 402 may be in communication with a data storage device (e.g., database 410, and/or the like), which may be local or remote to health database annotation system 402. In some embodiments, health database annotation system 402 may be capable of receiving information from, storing information in, communicating information to, or searching information stored in the data storage device. In some embodiments, health database annotation system 402 may be the same as or similar to computing device 100 and/or system and/or components pipeline 300, and other systems or devices that may perform the steps of method 200.

Computing device 404 may include one or more devices capable of receiving information and/or communicating information to health database annotation system 402, client device 406, server 408, and/or database 410 via communication network 412. For example, computing device 404 may include a computing device, such as a server, a group of servers, and/or other like devices. In some embodiments, computing device 404 may be associated with a server, a client device, and/or a user device as described herein.

Client device 406 may include one or more devices capable of receiving information from and/or communicating information to health database annotation system 402, computing device 404, server 408, and/or database 410 via communication network 412. Additionally or alternatively, one or more client devices 406 may include a device capable of receiving information from and/or communicating information to other client devices 406 via communication network 412, another network (e.g., an ad hoc network, a local network, a private network, a virtual private network, and/or the like), and/or any other suitable communication technique. For example, client device 406 may include a user device and/or the like.

Database 410 may include a computing device (e.g., a database device) configured to communicate with health database annotation system 402, computing device 404, client device 406, and/or server 408 via communication network 412. For example, database 410 may include a server, a group of servers, and/or other like devices. In some embodiments, database 410 may be associated with one or more computing devices providing interfaces such that a user may interact with database 410 via the one or more computing devices. Database 410 may be in communication with health database annotation system 402 such that database 410 is separate from health database annotation system 402. Alternatively, in some embodiments, database 410 may be part of (e.g., a component of) health database annotation system 402.

In some embodiments, database 410 may include a device capable of storing data (e.g., a storage device). In some embodiments, database 410 may include a collection of data stored and accessed by one or more computing devices. Database 410 may include file system storage, cloud storage, in-memory storage, and/or the like. Database 410 may include non-volatile storage (e.g., flash memory, magnetic media, and/or the like), volatile storage (e.g., random-access memory and/or the like), or both non-volatile and volatile storage. In some embodiments, database 410 may be part of (e.g., a component of) health database annotation system 402.

Communication network 412 may include one or more wired and/or wireless networks. For example, communication network 412 may include a cellular network (e.g., a long-term evolution (LTE®) network, a third generation (3G) network, a fourth generation (4G) network, a fifth generation (5G) network, a code division multiple access (CDMA) network, and/or the like), a public land mobile network (PLMN), a local area network (LAN), a wide area network (WAN), a metropolitan area network (MAN), a telephone network (e.g., the public switched telephone network (PSTN)), a private network (e.g., a private network associated with health database annotation system 402), an ad hoc network, an intranet, the Internet, a fiber optic-based network, a cloud computing network, and/or the like, and/or a combination of these or other types of networks.

The number and arrangement of systems, devices, and/or networks shown in FIG. 4 are provided as an example. There may be additional systems, devices, and/or networks; fewer systems, devices, and/or networks; different systems, devices, and/or networks; and/or differently arranged systems, devices, and/or networks than those shown in FIG. 4. Furthermore, two or more systems or devices shown in FIG. 4 may be implemented within a single system or device, or a single system or device shown in FIG. 4 may be implemented as multiple, distributed systems or devices. Additionally or alternatively, a set of systems (e.g., one or more systems) or a set of devices (e.g., one or more devices) of environment 400 may perform one or more functions described as being performed by another set of systems or another set of devices of environment 400.

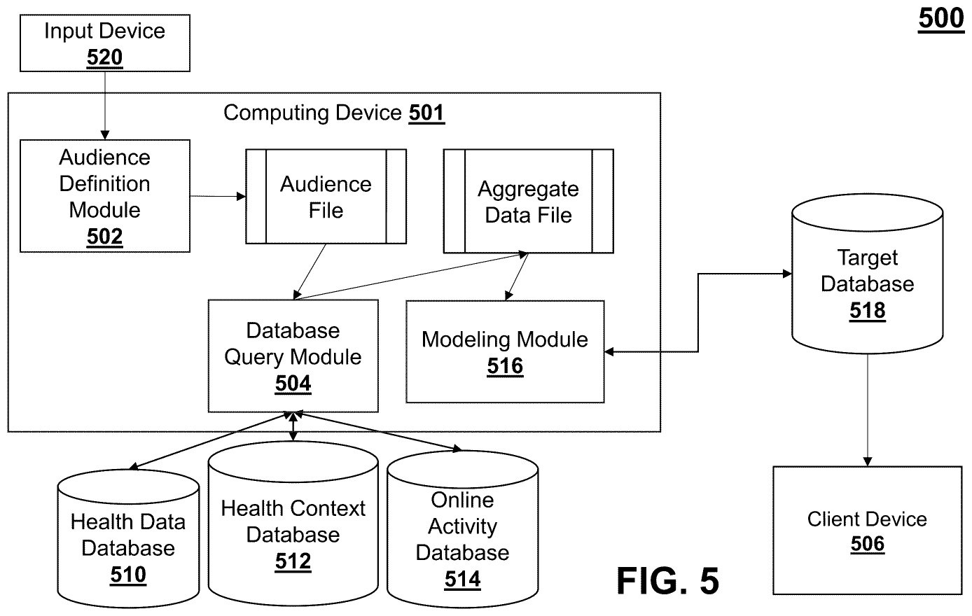

FIG. 5 shows a diagram of an exemplary architecture 500 for a computer system and/or network in which exemplary system configurations for generating and assessing data aggregations and/or annotating target databases may be implemented as disclosed herein. As shown in FIG. 5, architecture 500 can include computing device 501, audience definition module 502, database query module 504, client device 506, health data database 510, health context database 512, online activity database 514, modeling module 516, target database 518, and input device 520. Components and/or modules shown in FIG. 5 can be the same as or similar to components and/or modules shown and described with regard to FIG. 1.

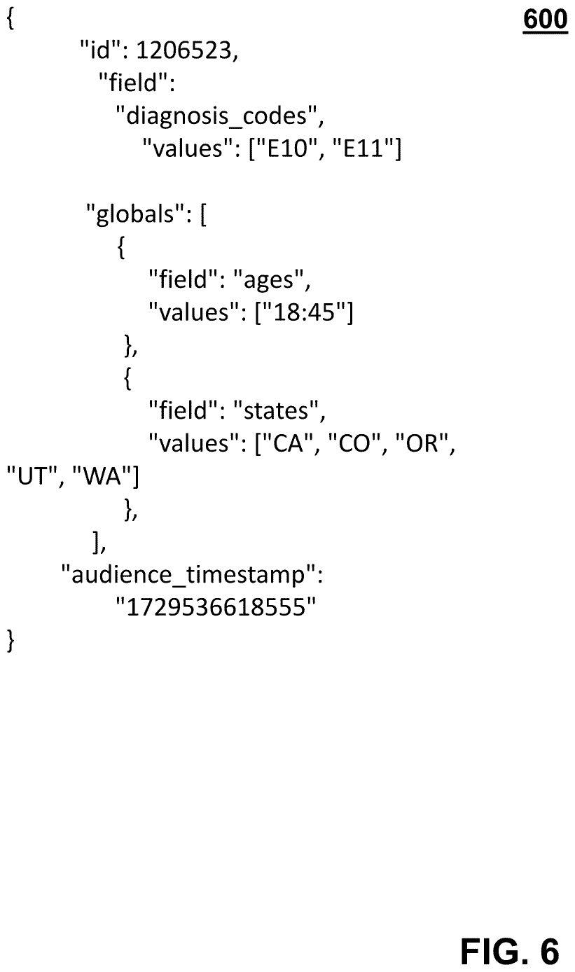

FIG. 6 shows a diagram of an exemplary audience file 600 including exemplary audience data objects for generating and assessing data aggregations and/or annotating target databases disclosed herein. As shown in FIG. 6, exemplary audience file 600 can include fields such as “ages”, “states” (e.g., a geographic location field), “diagnosis_codes” (e.g., a medical condition), an audience identifier and/or timestamp, and other data points.

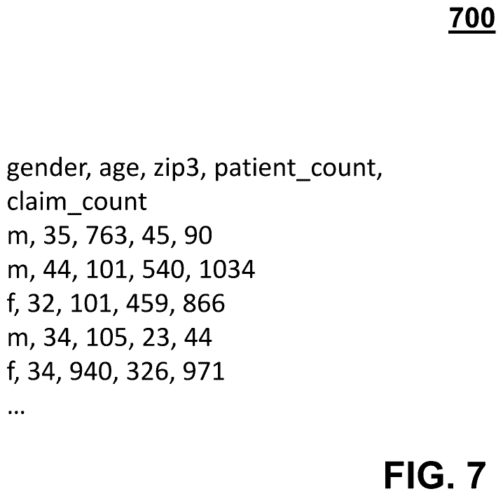

FIG. 7 shows a diagram of exemplary de-identified aggregate data 700 in an exemplary aggregate data file for generating and assessing data aggregations and/or annotating target databases as disclosed herein. As shown in FIG. 7, de-identified aggregate data may include at least a gender field, an age field, and/or a geographic location field (e.g., zip3). Each data entry may include a value indicating a patient count and/or a claim count for a patient and/or user data entry which contains a matching value when compared to other fields. For example, in the first row in FIG. 7, data entries having a gender value “m”, an age value “35”, and a zip3 value “763” had a patient count of “45” and a claim count of “90”, because processor 106 can count each data entry where the values of “m”, “35”, and “763” matched in the corresponding data fields.

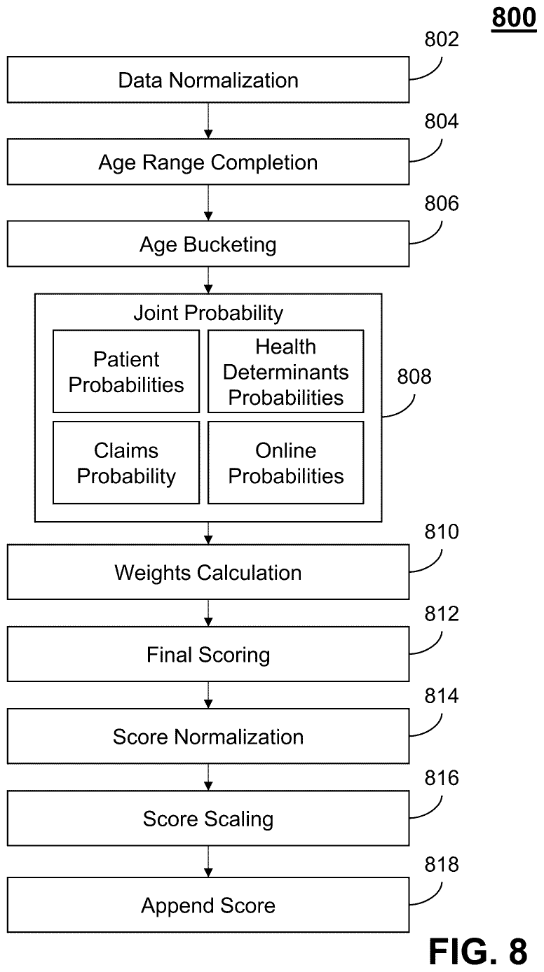

FIG. 8 is a flow diagram for an exemplary method 800 performed by a modeling module configured with program code for generating and assessing data aggregations and/or annotating target databases as disclosed herein. As shown in FIG. 8, method 800 may include step 802 of data normalization, step 804 of age range completion (e.g., data cleaning), step 806 of age bucketing (e.g., generating segments), step 808 of calculating a joint probability, including patient probabilities, social determinants of health probabilities, claims probabilities, and online probabilities. Method 800 may also include step 810 of weight calculations, step 812 of final scoring, step 814 of score normalization, step 816 of score scaling, and step 818 of appending the score to data entries in a database.

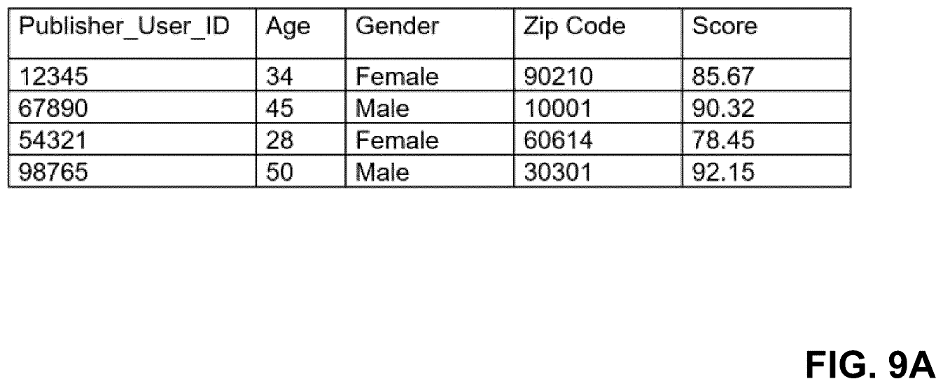

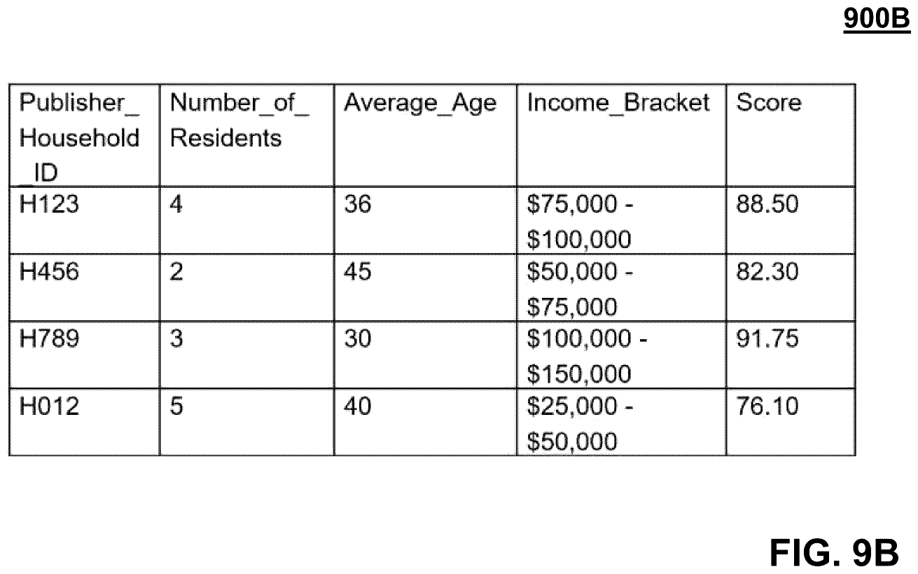

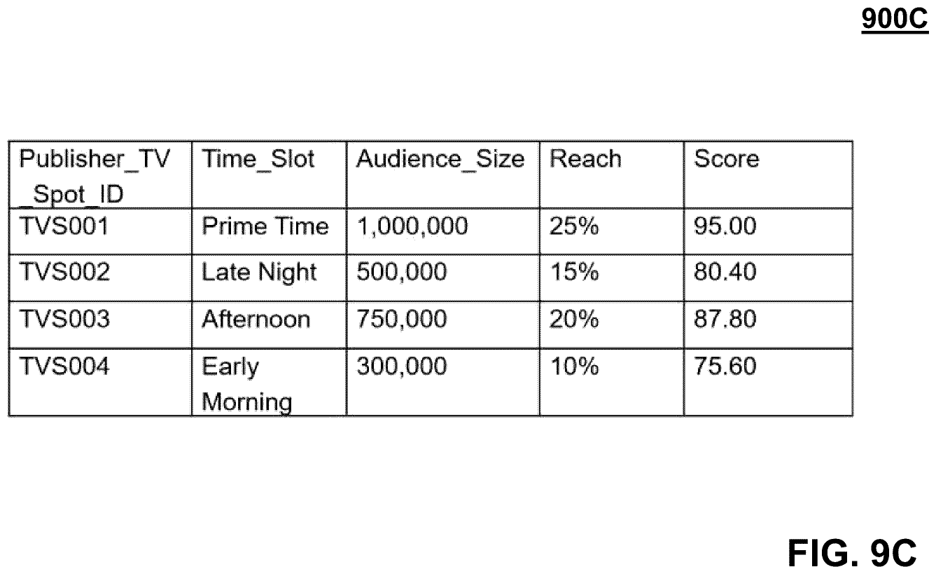

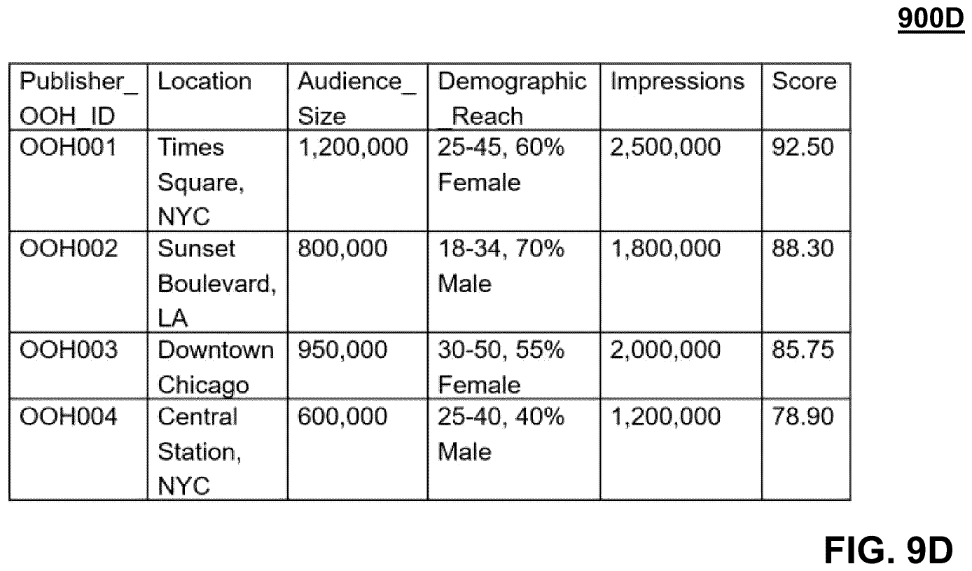

FIGS. 9A-9D show diagrams of exemplary database entries 900A-900D having appended final probability scores as disclosed herein. As shown in FIGS. 9A-9D, a final probability score may vary from values of 51 to 100. In some embodiments, data entries may include a publisher ID field that may identify at least one client device in which updated health information may be transmitted to and/or shared. Other various fields and/or values may be included in data entries, such as income or income bracket, number of residents, an average age, a time slot, an audience size, a reach percentage, and/or a number of impressions.

Any of the processors disclosed herein can include any integrated circuit or other electronic device (or collection of devices) capable of performing an operation on at least one instruction, which can include a Reduced Instruction Set Core (RISC) processor, a CISC microprocessor, a Microcontroller Unit (MCU), a CISC-based CPU, a DSP, a GPU, a Field Programmable Gate Array (FPGA), etc. The hardware of such devices can be integrated onto a single substrate (e.g., silicon “die”), or distributed among two or more substrates. Various functional aspects of the processor can be implemented solely as software or firmware associated with the processor.

The processor can include one or more processing or operating modules. A processing or operating module can be a software or firmware operating module configured to implement any of the functions disclosed herein. The processing or operating module can be embodied as software and stored in memory; the memory being operatively associated with the processor. A processing module can be embodied as a web application, a desktop application, a console application, etc.

The processor can include or be associated with a computer or machine readable medium. The computer or machine readable medium can include memory. Any of the memory discussed herein can be computer readable memory configured to store data. The memory can include a volatile or non-volatile, transitory or non-transitory memory, and be embodied as an in-memory, an active memory, a cloud memory, etc. Examples of memory can include flash memory, RAM, ROM, Programmable Read only Memory (PROM), Erasable Programmable Read only Memory (EPROM), Electronically Erasable Programmable Read only Memory (EEPROM), FLASH-EPROM, Compact Disc (CD)-ROM, Digital Optical Disc DVD), optical storage, optical medium, a carrier wave, magnetic cassettes, magnetic tape, magnetic disk storage or other magnetic storage devices, or any other medium which can be used to store the desired information and which can be accessed by the processor.

The memory can be a non-transitory computer-readable medium. The term “computer-readable medium” (or “machine-readable medium”) as used herein is an extensible term that refers to any medium or any memory, that participates in providing instructions to the processor for execution, or any mechanism for storing or transmitting information in a form readable by a machine (e.g., a computer). Such a medium can store computer-executable instructions to be executed by a processing element and/or control logic, and data which is manipulated by a processing element and/or control logic, and can take many forms, including but not limited to, non-volatile medium, volatile medium, transmission media, etc. The computer or machine readable medium can be configured to store one or more instructions thereon. The instructions can be in the form of algorithms, program logic, etc. that cause the processor to execute any of the functions disclosed herein.

Embodiments of the memory can include a processor module and other circuitry to allow for the transfer of data to and from the memory, which can include to and from other components of a communication system. This transfer can be via hardwire or wireless transmission. The communication system can include transceivers, which can be used in combination with switches, receivers, transmitters, routers, gateways, wave-guides, etc. to facilitate communications via a communication approach or protocol for controlled and coordinated signal transmission and processing to any other component or combination of components of the communication system. The transmission can be via a communication link. The communication link can be electronic-based, optical-based, opto-electronic-based, quantum-based, etc. Communications can be via Bluetooth, near field communications, cellular communications, telemetry communications, Internet communications, etc.

Data stored in the exemplary computing device (e.g., in the memory) can be stored on any type of suitable computer readable media, such as optical storage (e.g., a compact disc, digital versatile disc, Blu-ray disc, etc.), magnetic tape storage (e.g., a hard disk drive), or solid-state drive. An operating system can also be stored in the memory.

In an exemplary embodiment, the data can be configured in any type of suitable database configuration, such as a relational database, a structured query language (SQL) database, a distributed database, an object database, etc. Suitable configurations and storage types will be apparent to persons having skill in the relevant art.

The exemplary computing device can also include a communications interface. The communications interface can be configured to allow software and data to be transferred between the computing device and external devices. Exemplary communications interfaces can include a modem, a network interface (e.g., an Ethernet card), a communications port, a PCMCIA slot and card, etc. Software and data transferred via the communications interface can be in the form of signals, which can be electronic, electromagnetic, optical, or other signals as will be apparent to persons having skill in the relevant art. The signals can travel via a communications path, which can be configured to carry the signals and can be implemented using wire, cable, fiber optics, a phone line, a cellular phone link, a radio frequency link, etc. Transmission of data and signals can be via transmission media. Transmission media can include coaxial cables, copper wire, fiber optics, etc. Transmission media can also take the form of acoustic or light waves, such as those generated during radio-wave and infrared data communications, or other form of propagated signals (e.g., carrier waves, digital signals, etc.).

Memory semiconductors (e.g., DRAMs, etc.) can be means for providing software to the computing device. Computer programs (e.g., computer control logic) can be stored in the memory. Computer programs can also be received via the communications interface. Such computer programs, when executed, can enable computing devices to implement the present methods as discussed herein. In particular, the computer programs stored on a non-transitory computer-readable medium, when executed, can enable hardware processor devices to implement the methods as discussed herein. Accordingly, such computer programs can represent controllers of the computing device.

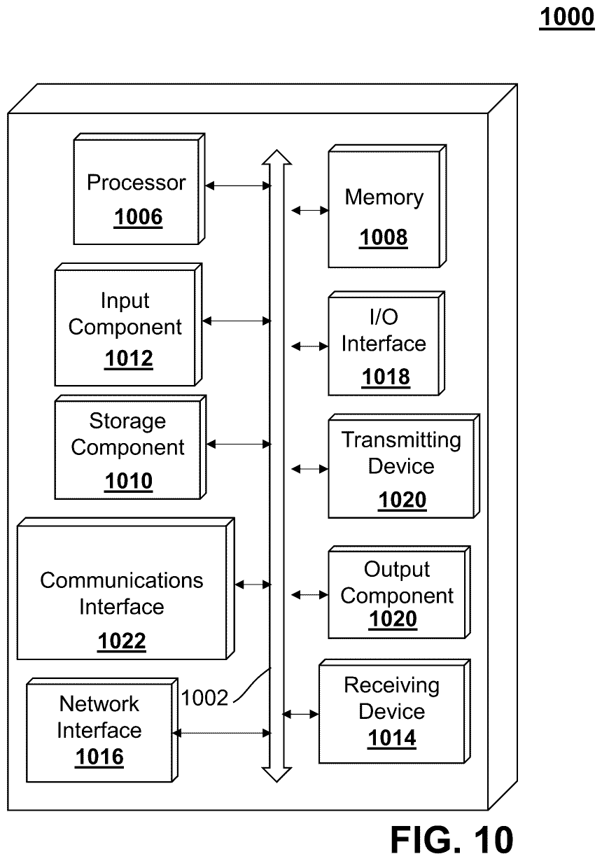

FIG. 10 shows a diagram of example components of a computing device or system 1000 as disclosed herein. Computing device 1000 (and/or at least one component of computing device 1000) can correspond to at least one of computing system 100, processor 106, memory 108, health data database 110, health context database 112, and/or online activity database 114 in FIG. 1 (and similarly to components for FIGS. 4 and 5). In some embodiments, such systems or devices in FIG. 1 can include at least one computing device 1000 and/or at least one component of computing device 1000. The number and arrangement of components shown in FIG. 10 are provided as an example. In some embodiments, computing device 1000 can include additional components, fewer components, different components, or differently arranged components than those shown in FIG. 10. Additionally, or alternatively, a set of components (e.g., one or more components) of computing device 1000 can perform one or more functions described as being performed by another set of components of computing device 1000.

Computing system or device 1000 can include processor 1006, memory 1008, storage component 1010, input component 1012, receiving device 1014, network interface 1016, input/output (I/O) interface 1018, transmitting device 1020, communications interface 1022, and communication infrastructure 1002. Memory 1008 can be the same as or similar to memory 108 as disclosed herein. Processor 1006 can be the same as or similar to processor 106 as disclosed herein.

Memory 1008 can be configured for storing program code for at least one module and/or at least one machine learning model. Memory 1008 can include one or more memory devices such as volatile or non-volatile memory. For example, the volatile memory can include random access memory. According to exemplary embodiments, the non-volatile memory can include one or more resident hardware components such as a hard disk drive and a removable storage drive (e.g., a floppy disk drive, a magnetic tape drive, an optical disk drive, a flash memory, or any other suitable device). The non-volatile memory can include an external memory device connected to communicate with system 1000 via a mobile communication network. According to an exemplary embodiment, an external memory device can be used in place of any resident memory devices. Data stored in system 1000 can be stored on any type of suitable computer readable media, such as optical storage (e.g., a compact disc, digital versatile disc, Blu-ray disc, etc.) or magnetic tape storage (e.g., a hard disk drive). The stored data can include network traffic data, log data, streaming events, and/or CDRs generated and/or accessed by processor 1006, and software or program code used by processor 1006 for performing the tasks associated with the exemplary embodiments described herein. The data can be configured in any type of suitable database configuration, such as a relational database, a structured query language (SQL) database, a distributed database, an object database, etc. Suitable configurations and storage types will be apparent to persons having skill in the relevant art.

Receiving device 1014 can be a combination of hardware and software components configured to receive data samples from the mobile network or database. According to exemplary embodiments, receiving device 1014 can include a hardware component such as an antenna, a network interface (e.g., an Ethernet card), a communications port, a Personal Computer Memory Card International Association (PCMCIA) slot and card, 5G New Radio (NR) interface, or any other component or device suitable for use on a mobile communication network or Radio Access Network as desired. Receiving device 1014 can be an input device for receiving signals and/or data samples formatted according to 3GPP protocols and/or standards. Receiving device 1014 can be connected to other devices via a wired or wireless network or via a wired or wireless direct link or peer-to-peer connection without an intermediate device or access point. The hardware and software components of receiving device 1014 can be configured to receive the data from the mobile network according to one or more communication protocols and data formats. For example, receiving device 1014 can be configured to communicate over a network, which can include a LAN, a WAN, a wireless network (e.g., Wi-Fi), a mobile communication network, a satellite network, the Internet, fiber optic cable, coaxial cable, infrared, radio frequency (RF), another suitable communication medium as desired, or any combination thereof. During a receive operation, receiving device 1014 can be configured to identify parts of the received data via a header and parse the data signal and/or data packet into small frames (e.g., bytes, words) or segments for further processing at processor 1006.

Processor 1006 can be configured for executing the program code stored in memory 1008. Processor 1006 can be a special purpose or a general purpose computing device encoded with program code or software for performing the exemplary functions and/or features disclosed herein. According to exemplary embodiments of the present disclosure, processor 1006 can include a CPU. The CPU can be connected to the communications infrastructure including a bus, message queue, or network, multi-core message-passing scheme, for communicating with other components of computing system 1000, such as memory 1008, input component 1012, communications interface 1022, and I/O interface 1018. The CPU can include one or more processors such as a microprocessor, microcomputer, programmable logic unit or any other suitable hardware computing devices as desired.

I/O interface 1018 can be configured to receive the signal from processor 1006 and generate an output suitable for a peripheral device via a direct wired or wireless link. I/O interface 1018 can include a combination of hardware and software for example, a processor, circuit card, or any other suitable hardware device encoded with program code, software, and/or firmware for communicating with a peripheral device such as a display device, printer, audio output device, or other suitable electronic device or output type as desired.

Transmitting device 1020 can be configured to receive data from processor 1006 and assemble the data into a data signal and/or data packets according to the specified communication protocol and data format of a peripheral device or remote device to which the data is to be sent. Transmitting device 1020 can include any one or more of hardware and software components for generating and communicating the data signal over communications infrastructure 1002 and/or via a direct wired or wireless link to a peripheral or remote device. Transmitting device 1020 can be configured to transmit information according to one or more communication protocols and data formats as discussed in connection with receiving device 1014.

According to exemplary embodiments described herein, memory 1008 and processor 1006 can store and/or execute computer program code for performing the specialized functions described herein. It should be understood that the program code can be stored on a non-transitory computer usable medium, such as memory devices for system 1000 (e.g., computing device), which can be memory semiconductors (e.g., DRAMs, etc.) or other tangible non-transitory means for providing software to system 1000. The computer programs (e.g., computer control logic) or software can be stored in memory devices (e.g., device memory 1008) resident on/in system 1000. The computer programs can also be received from external storage devices and/or network storage locations via a communications interface. Such computer programs, when executed, can enable system 1000 to implement the present methods and exemplary embodiments discussed herein. Accordingly, such computer programs can represent controllers of system 1000. Where the present disclosure is implemented using software, the software can be stored in a computer program product or non-transitory computer readable medium and loaded into system 1000 using any one or combination of a removable storage drive, an interface for internal or external communication, and a hard disk drive, where applicable.

In the context of exemplary embodiments of the present disclosure, a processor can include one or more modules or engines configured to perform the functions of the exemplary embodiments described herein. Each of the modules or engines can be implemented using hardware and, in some instances, can also utilize software, such as corresponding to program code and/or programs stored in memory. In such instances, program code can be interpreted or compiled by the respective processors (e.g., by a compiling module or engine) prior to execution. For example, the program code can be source code written in a programming language that is translated into a lower level language, such as assembly language or machine code, for execution by the one or more processors and/or any additional hardware components. The process of compiling can include the use of lexical analysis, preprocessing, parsing, semantic analysis, syntax-directed translation, code generation, code optimization, and any other techniques that can be suitable for translation of program code into a lower level language suitable for controlling system 1000 to perform the functions disclosed herein. It will be apparent to persons having skill in the relevant art that such processes result in system 1000 being a specially configured computing device uniquely programmed to perform the functions of the exemplary embodiments described herein.

It will be appreciated by those skilled in the art that the present invention can be embodied in other specific forms without departing from the spirit or essential characteristics thereof. The presently disclosed embodiments are therefore considered in all respects to be illustrative and not restrictive. The scope of the invention is indicated by the appended claims rather than the foregoing description and all changes that come within the meaning and range and equivalence thereof are intended to be embraced therein.

What is claimed is:

1. A computer-implemented method for generating and assessing data aggregations, the method comprising:
defining audience characteristic data, wherein the audience characteristic data comprises any combination of a geographic location data, demographic data, and/or a medical condition;
querying a health data database including health data entries, each health data entry including at least an age field, a gender field, and a geographic location field, wherein the audience characteristic data is received as health information comprised of a set of individual data points paired with an age value, a gender character, and a geographic location value which is correlated with the health information;
querying a health context database, including health context data entries, each health context data entry including at least an age field, a gender field, and a geographic location field, wherein the audience characteristic data is received as health context information comprised of a set of individual data points paired with an age value, a gender character, and a geographic location value which is correlated with the health context information;
aggregating the health information received from the health data database, the health context information received from the health context database, into a set of de-identified audience data segments organized by age, gender, and geographic location,
wherein the set of de-identified audience data segments is devoid of personally identifiable information (PII),
and wherein each de-identified audience data segment in the set includes at least a count for the segment and values for the individual data points;
assigning each of the de-identified audience data segments of the set to an age bucket within a set of age buckets, the age buckets defined by an age range and a gender;
calculating a probability distribution for the set of age buckets;
calculating a probability score for each of the age buckets in the set using the probability distribution and weights assigned to the individual data points;
querying a target database, keyed by at least age, gender, and geographic location, and appending the calculated probability score of the age bucket to one or more target data entries in the target database that matches with the age, gender, and geographic information from the target database; and
providing an interface to updated health information associated with the calculated probability score and at least one audience data segment of the set of de-identified audience data segments such that the updated health information is accessible to the target database based on relevance to client devices associated with target entries in the target database, wherein the updated health information is not associated with any PII.

2. The method of claim 1, further comprising transmitting the updated health information to one or more client devices associated with at least one audience data segment of the set of de-identified audience data segments such that the updated health information is targeted based on relevance to users of the one or more client devices without being associated to any PII.

3. The method of claim 1, further comprising responding to a request from the target database for the updated health information by transmitting updated health information to the target database.

4. The method of claim 1, further comprising
assigning each of the de-identified audience data segments of the set to any one or more of a claim count bucket within a set of claim count buckets, a gender bucket within a set of gender buckets, a social determinant of health bucket within a set of social determinants of health buckets, and a patient bucket within a set of patient buckets, the claim count buckets defined by a claim count range and a gender; the gender bucket defined by a gender identifier, the social determinant of health bucket defined by a context attribute, and the patient bucket defined by a range of patient counts;
calculating a probability distribution for any one or more of the set of claim count buckets, the set of gender buckets, the set of social determinants of health buckets, and the set of patient buckets; and
calculating a probability score for each bucket in each set using the probability distribution and weights assigned to the individual data points.

5. The method of claim 1, wherein the querying of the health data database further comprises limiting the received health information to entries corresponding to the medical condition of the audience characteristic data.

6. The method of claim 5, wherein the medical condition is defined using any one or more of an ICD-10 code, a National Drug Code (NDC), a current procedural terminology (CPT), or an age value.

7. The method of claim 1, wherein the audience characteristic data further comprises at least one of a set of geographic regions, a prescribed treatment, and insurance coverage information.

8. The method of claim 1, wherein the assigning each of the de-identified audience data segments of the set to an age bucket within a set of age buckets is performed dynamically using a clustering technique driven by age to identify optimal split points based on counts in the de-identified audience data segments.

9. The method of claim 1, wherein the health data database contains information concerning medical claims, treatment histories, prescription information, and other healthcare information.

10. The method of claim 1, wherein the health context database contains information concerning socioeconomic status, education, occupation, marital status, household income, education level, ethnicity, occupation, family structure, and homeownership status.

11. The method of claim 4, wherein the calculation of the probability score for each of the buckets further comprises initially normalizing the probability scores for each of the buckets using a square root transformation to apply a mathematical spread to highlight differences between the buckets, and subsequently scaling the normalized probability scores for each of the buckets to arrive at probabilities between 51 and 100.

12. The method of claim 1, wherein the target database includes individual entries for a unique user.

13. The method of claim 1, wherein the target database includes individual entries for a unique television spot defined at least by a time slot and an audience size.

14. The method of claim 1, wherein the target database includes individual entries for an out of home placement defined at least by a physical location, an audience size, and a demographic reach.

15. The method of claim 14, wherein the individual entries for an out of home placement are selected from the group consisting of a billboard advertisement along a roadway, an advertisement in a shopping mall, an advertisement in a public transit system, or an advertisement at a sporting venue.

16. The method of claim 1, further comprising querying an online activity database including online activity data entries, each online activity data entry including at least an age, a gender, and a geographic location field, wherein the audience characteristic data is received as online activity information comprised of a set of individual data points paired with an age value, a gender character, and a geographic location value which is correlated with the online activity information.

17. The method of claim 16, wherein the online activity database contains information collected from website interactions including demographic information relating to anonymized user interaction with the website from which the information was collected.

18. A system for managing data aggregations and annotating target databases, comprising:
at least one health data database and at least one health context database;
a memory storing program code for an audience definition module, a database query module, and a modeling module;
a processor configured to execute program code, wherein the program code for the audience definition module, when executed, will cause the processor to:
receive text input defining an audience definition, wherein the text input includes at least an age range and a medical condition; and
structure the text input into an audience data object in an audience file including at least an age range field and a medical condition field;
wherein the program code for the database query module, when executed, will cause the processor to:
query a health data database to extract corresponding health data entries based on the audience file;
query a health context database to extract corresponding health context data entries based on the audience file;
synthesize the extracted corresponding health data entries and corresponding health context data entries, wherein the corresponding health data entries and the corresponding health context data entries do not include any personally identifiable information (PII) to generate de-identified aggregate data in an aggregate data file;
wherein the program code for the modeling module, when executed, will cause the processor to:
clean the de-identified aggregate data to generate consistent data fields within the aggregate data file;
automatically cluster the de-identified aggregate data into plural segments based on at least one parameter;
calculate plural joint probability values based on the plural segments, and the aggregate data;
calculate a final probability score for each of the plural segments using the plural joint probability values and weights assigned to each field in the audience data objects to generate an indexing model;
query a target database, including data entries with at least an age field, a gender field, and a geographic location field; and
append each of the final probability scores calculated for each of the plural segments to a target data entry in the target database, wherein the segment matches with the age field, the gender field, and the geographic information field from the target data entry in the target database.

19. The system of claim 18, wherein the program code for the modeling module, when executed, will cause the processor to transmit updated health information to the target database for transmitting to one or more client devices associated with at least one segment such that the updated health information is transmitted to users where the updated health information is useful to the client devices without being associated to any PII of the users.

20. The system of claim 18, further comprising at least one online activity database, wherein the program code for the modeling module, when executed, will cause the processor to query an online activity database to extract corresponding online activity data entries based on the audience file;
wherein when the program code for the modeling module, when executed, causes the processor to synthesize the extracted corresponding health data entries, the program code for the modeling module will cause the processor to synthesize corresponding online activity data entries with the corresponding health data entries and the corresponding health context data entries, wherein the corresponding online activity data entries do not include any PII.
